(19) United States
(12) Patent Application Publication
(10) Pub. No.: US 2026/0106005 A1
(43) Pub. Date: Apr. 16, 2026

(54) UNIVERSAL MEDICAL CHARTING

(71) Applicant: CERNER INNOVATION, INC., Kansas City, MO (US)

(72) Inventors: Joel Isernhagen, Overland Park, KS (US); Rob Helton, Shawnee, KS (US); Missy Brooks, Washington, DC (US); Anna Terranella, Kansas City, KS (US); Jessica Kalwei, Kansas City, MO (US); Nick Kroeker, Centennial, CO (US)

(73) Assignee: CERNER INNOVATION, INC., Kansas City, MO (US)

(21) Appl. No.: 19/421,716

(22) Filed: Dec. 16, 2025

Related U.S. Application Data
(63) Continuation of application No. 16/728,921, filed on Dec. 27, 2019.
(60) Provisional application No. 62/786,927, filed on Dec. 31, 2018.

(51) Int. Cl. G16H 15/00; G16H 10/60; G16H 20/30

(52) U.S. Cl. CPC G16H 15/00; G16H 10/60; G16H 20/30

(57) ABSTRACT

A cloud-based integrated charting system enables generation and display of customizable medical charts. Upon receiving a request from a client device, the system accesses a database of universal charting elements, incorporates user-provided configuration input, and applies region-specific rules to generate chart data. The resulting medical chart is displayed on the client device and stored with regional context. The system further refines its charting capabilities by training a machine learning model using the generated chart data, allowing subsequent chart requests from similarly located devices to benefit from regionally relevant chart customization.

### CROSS-REFERENCE TO RELATED APPLICATIONS AND PRIORITY CLAIM

This application is a continuation of U.S. Ser. No. 16/728,921, filed Dec. 27, 2019, entitled “Universal Medical Charting,” which claims the benefit of U.S. Provisional Ser. No. 62/786,927 , filed Dec. 31, 2018, entitled “Universal Medical Charting,” the entire contents of which are incorporated herein by reference.

### BACKGROUND

An electronic medical chart is a record of a patient's key clinical data and medical history, such as vital signs, diagnoses, conditions, demographic information medications, treatment plans, progress notes, problems, immunization dates, allergies, radiology images, and laboratory and test results.

A medical chart includes medical notes made by a physician, nurse, lab technician or any other member of a patient's medical team. Accurate and complete medical charts ensure systematic documentation of a patient's medical history, diagnosis, treatment and care.

In computerized medical charting, current charting views are static and present static data elements to a provider which may not be relevant to the patient in the charting context. When a medical provider accesses a person's electronic medical chart to perform medical charting in a computer environment, the charting view is static. For example, when vital signs are to be charted for a patient, a static view with multiple vital signs is presented to the medical provider. The medical charting form is static and inflexible and presents the same things to be charted regardless of patient, condition and physician. Currently, thousands of static medical charts include thousands of static sections for hundreds of medical departments. However, there is no universal relationship of the data elements in the static charts and sections.

This results in disconnect between medical charting data elements utilized by different departments and clients. For example, the vital signs charting data elements documented for a patient by an emergency department in an institution are not universally the same as vital signs charting data elements documented in an ambulatory setting within the same institution. As such, even the vital signs charting data elements within the same institution differ and updates to the charting elements has to be done on an individual department basis. In addition, it is difficult to map and track the charting data elements between departments and even more costly and time-consuming to do so between different institutions.

### BRIEF SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Embodiments of the present disclosure relate to systems, methods, and user interfaces for providing a medical chart building and tracking across departments and clients using a universal library of data elements for medical charting. More particularly, embodiments of the present disclosure provide a cloud-based integrated charting system that utilizes a universal medical charting build tool and universal library of data elements across departments and clients to provide insights and model medical charting builds.

To do so, a universal library of data elements and medical charting build tool is provided that is accessible by clients of the provider. The universal library of data elements and charting build tool has access to a centralized concept mapping service that collects client medical charting build data and creates model charting builds corresponding to patient condition, provider and location.

When a request is received from a user of the client via a device to build medical charting for the client, the client corresponding to the user is identified. Access to the medical charting build tool and universal library of data elements provided to the client user via the device. The medical charting build tool and universal library of data elements enables the client user to build charts for the client based on patient conditions, providers and locations. The medical charting build tool allows the user to create a medical charting form that a clinician utilizes while charting medical data for a patient. The client user can decide what data elements to display and hide when the chart is rendered or displayed to a clinician. The clinician then fills in the value or answer for the data elements displayed in the charting form (e.g., numeric blood pressure, alphanumeric answer or selection of options).

As medical charting forms are created, the data is stored in the client database and transmitted to the medical charting build tool for analysis and development of model medical charting forms. In embodiments, the user can build medical charting forms based on patient condition, provider and location. These modifications can be learned by the cloud-based integrated charting system, utilizing machine learning techniques.

### BRIEF DESCRIPTION OF THE SEVERAL VIEWS OF THE DRAWINGS

The present invention is described in detail below with reference to the attached drawing figures, wherein:

FIG. 1 is a block diagram of an exemplary operating environment suitable to Implement embodiments of the present invention;

FIG. 2 depicts an exemplary framework of a cloud-based integrated medical charting build tool suitable to implement embodiments of the present invention;

FIG. 3 depicts an exemplary hierarchy framework of universal medical charting data elements utilized by cloud-based medical charting build tool suitable to implement embodiments of the present invention;

FIG. 4 depicts an exemplary hierarchy framework and screen display of universal medical charting data elements utilized by cloud-based medical charting build tool wherein some of the data elements have been selected by a client to be displayed in a medical chart for a critical care departments and some have been selected to be hidden in an embodiment of the present invention;

FIG. 5 depicts an exemplary hierarchy framework and screen display of universal medical charting data elements utilized by cloud-based medical charting build tool wherein some of the data elements have been selected by a client to be displayed in a medical chart form for a physical therapy department and some have been selected to be hidden in an embodiment of the present invention;

FIG. 6 depicts a flow diagram of a method for providing a client user access to the medical charting build tool, universal medical charting data elements and rules engine for building medical charting forms for the client based on patient condition, location and providers, in accordance with an embodiment of the present invention; and

FIG. 7 depicts a flow diagram of a method for creating and transmitting model medical charting forms, in accordance with embodiments of the invention.

### DETAILED DESCRIPTION

The subject matter of the present invention is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” might be used herein to connote different elements of methods employed, the terms should not be interpreted as implying any particular order among or between various steps herein disclosed unless and except when the order of individual steps is explicitly stated.

Currently charting views are static. When a medical provider accesses a person's electronic medical record to perform medical charting in a computer environment, the charting view is static. For example, when vital signs are to be charted for a patient, a static view with all vital signs is presented to the medical provider. The medical charting form is static and presents the same questions regardless of patient condition, location and/or clinician. Current medical charts include thousands of sections for hundreds of medical departments, but no relationship between the sections and departments.

Embodiments of the present invention provide smart charting for medical providers. Based on conditionality, the charting questions presented to a medical provider are based on conditionality based on the context of the provider and specific type of encounter. Based on the condition, location and/or provider, the user interface presents only needed charting questions in the context of the provider and diagnosis for the encounter. The clinician does not have to look at excess charting elements and screen real estate space is preserved for charting elements important to the encounter and provider. Embodiments of the present invention create a medical charting experience for a charting that exposes the minimum data set necessary for a clinician to chart values for a patient.

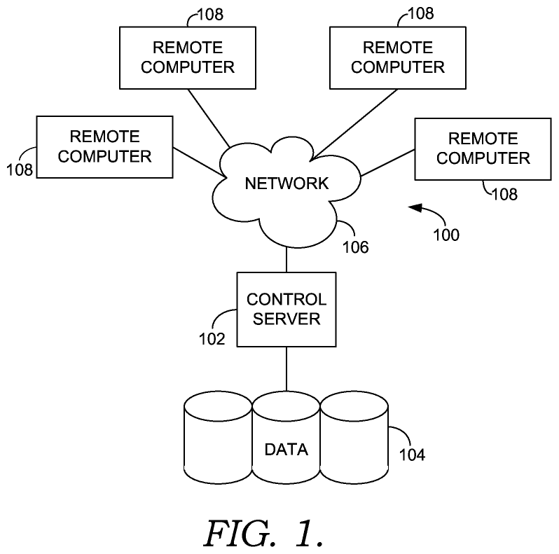

Having briefly described embodiments of the present invention, an exemplary operating environment suitable for use in implementing embodiments of the present invention is described below. FIG. 1 provides an aspect of an example operating environment with which embodiments of the present invention may be implemented. The aspect of an operating environment is illustrated and designated generally as reference numeral 100.

Example operating environment 100 comprises a general purpose computing device in the form of a control server 102. Exemplary components of the control server 102 comprise a processing unit, internal system memory, and a suitable system bus for coupling various system components, including database cluster 104, with the control server 102. The system bus might be any of several types of bus structures, including a memory bus or memory controller, a peripheral bus, and a local bus, using any of a variety of bus architectures. Exemplary architectures comprise Industry Standard Architecture (ISA) bus, Micro Channel Architecture (MCA) bus, Enhanced ISA (EISA) bus, Video Electronic Standards Association (VESA) local bus, and Peripheral Component Interconnect (PCI) bus, also known as Mezzanine bus.

Control server 102 typically includes therein, or has access to, a variety of computer-readable media, for instance, database cluster 104. Computer-readable media can be any available media that might be accessed by control server 102, and includes volatile and nonvolatile media, as well as, removable and nonremovable media. Computer-readable media might include computer storage media. Computer storage media includes volatile and nonvolatile media, as well as removable and nonremovable media implemented in any method or technology for storage of information, such as computer-readable instructions, data structures, program modules, or other data. In this regard, computer storage media might comprise RAM, ROM, EEPROM, flash memory or other memory technology, CD-ROM, digital versatile disks (DVDs) or other optical disk storage, magnetic cassettes, magnetic tape, magnetic disk storage, or other magnetic storage device, or any other medium which can be used to store the desired information and which may be accessed by the control server 102. Computer storage media does not comprise signals per se. Combinations of any of the above also may be included within the scope of computer-readable media.

The computer storage media discussed above and illustrated in FIG. 1, including database cluster 104, provide storage of computer-readable instructions, data structures, program modules, and other data for the control server 102. In some embodiments, database cluster 104 takes the form of a cloud-based data store, and in some embodiments is accessible by a cloud-based computing platform.

The control server 102 might operate in a computer network 106 using logical connections to one or more remote computers 108. Remote computers 108 might be located at a variety of locations in a medical or research environment, including clinical laboratories (e.g., molecular diagnostic laboratories), hospitals and other inpatient settings, veterinary environments, ambulatory settings, medical billing and financial offices, hospital administration settings, home healthcare environments, and providers' offices. Providers may comprise a treating physician or physicians; specialists such as surgeons, radiologists, cardiologists, and oncologists; emergency medical technicians; physicians' assistants; nurse practitioners; nurses; nurses' aides; pharmacists; dieticians; microbiologists; laboratory experts; laboratory technologists; genetic counselors; researchers; veterinarians; students; and the like.

The remote computers 108 might also be physically located in nontraditional medical care environments so that the entire healthcare community might be capable of integration on the network. The remote computers 108 might be personal computers, servers, routers, network PCs, peer devices, other common network nodes, or the like and might comprise some or all of the elements described above in relation to the control server 102. The devices can be personal digital assistants or other like devices.

Exemplary computer networks 106 comprise local area networks (LANs) and/or wide area networks (WANs). Such networking environments are commonplace in offices, enterprise-wide computer networks, intranets, and the Internet. When utilized in a WAN networking environment, the control server 102 might comprise a modem or other means for establishing communications over the WAN, such as the Internet. In a networked environment, program modules or portions thereof might be stored in association with the control server 102, the database cluster 104, or any of the remote computers 108. For example, various application programs may reside on the memory associated with any one or more of the remote computers 108. It will be appreciated by those of ordinary skill in the art that the network connections shown are exemplary and other means of establishing a communications link between the computers (e.g., control server 102 and remote computers 108) might be utilized.

In operation, an organization might enter commands and information into the control server 102 or convey the commands and information to the control server 102 via one or more of the remote computers 108 through input devices, such as a keyboard, a pointing device (commonly referred to as a mouse), a trackball, or a touch pad. Other input devices comprise microphones, satellite dishes, scanners, or the like. Commands and information might also be sent directly from a remote healthcare device to the control server 102. In addition to a monitor, the control server 102 and/or remote computers 108 might comprise other peripheral output devices, such as speakers and a printer.

In some embodiments, control server 102 is a computing system or platform made up of one or more computing devices. Embodiments of control server 102 may be a distributed computing system, a centralized computing system, a single computer such as a desktop or laptop computer or a networked computing system. Thus, in some embodiments, control server 102 comprises a multi-agent computer system with software agents.

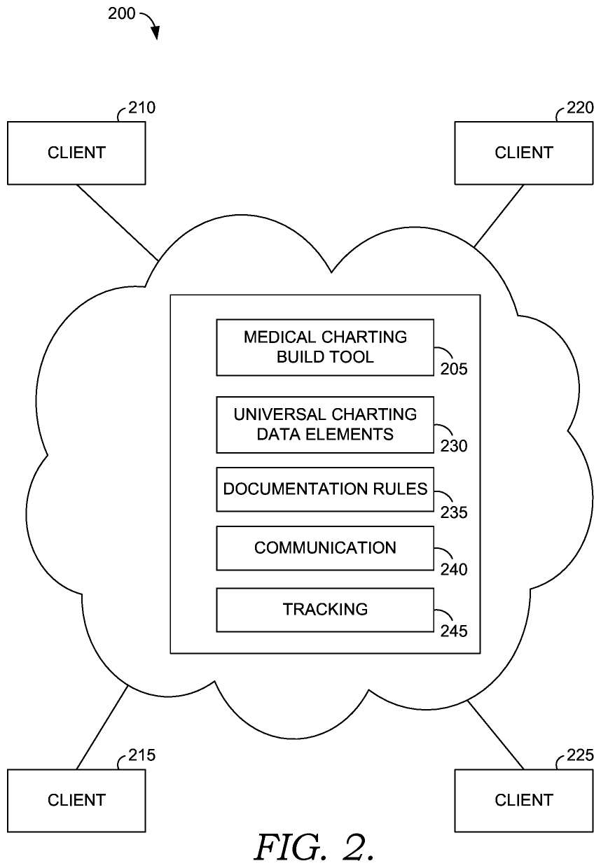

Turning now to FIG. 2, an exemplary framework of a cloud-based integrated charting system 200 is shown, in accordance with an aspect of the present invention. It should be understood that this and other arrangements described herein are set forth only as examples.

Other arrangements and elements (e.g., machines, interfaces, functions, orders, and groupings of functions, etc.) can be used in addition to or instead of those shown, and some elements may be omitted altogether. Further, many of the elements described herein are functional entities that may be implemented as discrete or distributed components or in conjunction with other components, and in any suitable combination and location. Various functions described herein as being performed by one or more entities may be carried out by hardware, firmware, and/or software. For instance, various functions may be carried out by a processor executing instructions stored in memory. The cloud-based integrated charting system 200 may be implemented via any type of computing device, such as computing device 100 described above with reference to FIG. 1, for example.

The cloud-based integrated charting system 200 includes medical charting build tool 205, universal charting data elements 230, documentation rules engine 235, communication module 240, tracking module 245, and user interface 250. It should be understood that the cloud-based integrated charting system 200 shown in FIG. 2 is an example of one suitable computing system architecture. Each of the components shown in FIG. 2 may be implemented via any type of computing device, such as computing device 100 described with reference to FIG. 1, for example.

The components may communicate with each other via a network, which may include, without limitation, one or more local area networks (LANs) and/or wide area networks (WANs). Such networking environments are commonplace in offices, enterprise-wide computer networks, intranets, and the Internet. It should be understood that any number provider and client databases may be employed within the cloud-based integrated charting system 200 within the scope of the present disclosure. Each may comprise a single device or multiple devices cooperating in a distributed environment. Additionally, other components not shown may also be included within the network environment.

Generally, medical charting build tool 205 and universal charting data elements 230 provide a central lookup service and universal tool for clients to build medical charting forms within the cloud-based integrated charting system 200. To do so, the tracking module 245 tracks client medical charting forms built utilizing universal charting data elements 230 corresponding to client domain specific customized builds. This enables the system 200 to keep track of the universal charting data elements 230 being used and customized medical charting builds across clients 210-225.

The universal charting data elements database 230 generally stores universal medical charting elements. Charting values by a clinician include patient's key clinical data and medical history, such as vital signs, diagnoses, conditions, demographic information medications, treatment plans, progress notes, problems, immunization dates, allergies, radiology images, and laboratory and test results. The charting elements may be questions (such as heart rate).

The universal charting data elements database 230 is a universal multi-use content library to be used by all clients 210, 215, 220 and 225 in an electronic medical record system. It will be appreciated that clients may be different medical institutions and/or departments may include different departments within a client institution.

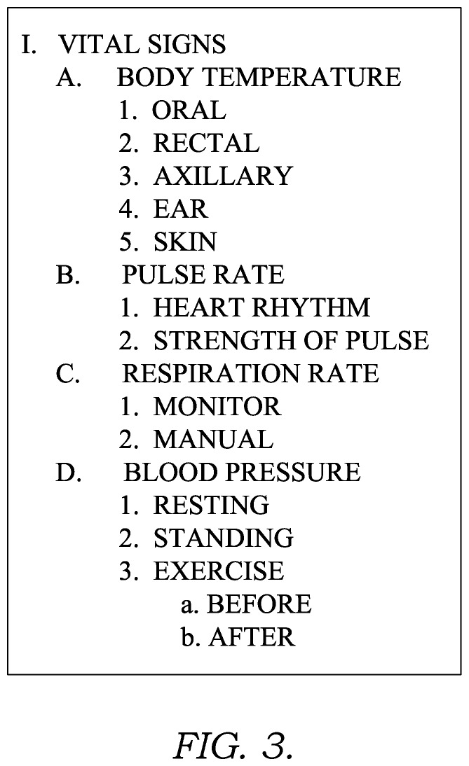

The universal charting data elements 230 includes medical charting data elements that are hard-coded to cloud-based integrated charting system 200 and cannot be changed during use by a client of the medical charting build tool 205. The universal charting data elements 230 are maintained in a hierarchical structure. For example, with reference to FIG. 3, all medical charting data elements for vital signs are in an event set hierarchy that creates relationships between hard-coded data elements. In the example with reference to FIG. 3, primary vital signs charting data elements include secondary elements (A-D) of body temperature, pulse rate, respiration rate and blood pressure. Each of the secondary elements contains tertiary elements. For example, secondary charting data element body temperature (A) includes tertiary elements (1-5) of oral, rectal axillary, ear and skin. It is pristine content in that universal charting data elements 230 of system 200 can be utilized to by multiple clients 210-225.

Additional primary, secondary and tertiary elements can be added to the multi-use content library by a universal data element repository database administrator. When a client makes a change to a medical data charting element, this information is tracked by a tracking application but does not create any hard-coded changes to the universal charting data elements database 230.

Clients utilize medical charting build tool 205 and universal charting data elements 230 utilizing web exposed service, such as Alva. A client content builder utilizes the medical charting build tool 205 to access the universal charting data elements 230 by selecting universal charting data elements 230 for medical charting forms. The client content builder can choose to expose or display certain universal charting data elements 230 while choosing to hide other charting data elements. The client content builder builds medical charting forms specific to any combination of the type of patient encounter (ED, Physical Therapy), the context of the provider (e.g., cardiologist for heart failure) and condition of the patient.

System 200 utilizes documentation rules engine 235 includes conditionality rules are applied to deploy best medical charting practices among clients. Documentation rules 236 include conditional rules applied when utilizing the medical charting build tool 205 to build medical charting forms. The documentation rules 236 include regulatory rules-for example every region has different Medicaid, Medicare, bundle payments and MACRA and behavioral health requirements so a client can build out medical charting forms for what is needed to their region. Should a client build a medical charting form that excludes important regulatory requirements, the documentation rules engine alerts the builder that medical chart built is not compliant with federal, state or county regulations. In addition to applying regulatory logic to during client build, meaningful use rules and requirements by the client institution can also be applied to the chart build notifying the client that meaningful use requirements are missing in the medical chart build. The client builder then adds the missing data elements to satisfy the conditionality rules.

System 200 is deployed in a cloud environment so that best medical charting practices can be tracked by tracking module 245 of the cloud-based integrated charting system 200. The tracking module 245 curates main use cases, hard codes the cases with hard-coded medical charting elements. Additionally, the application tracks when a client copies content and applies elsewhere within the build.

Tracking module 245 brings back the best of medical chart build to the universal charting data elements database 230. The tracking module includes type of charting based on condition, provider and/or location and client domain specific charting preference builds. The medical chart builds can be utilized by the system 200 to assist in chart building and deploying for other clients. System 200 enable the provider to create and curate various model charting that can be reconciled with the client domain specific needs. For example, a client operating in the same or similar location as another client who has already built a medical chart for that location may be able to utilize the medical chart build developed by the client in the same or similar location. The client utilizing the other client's medical chart build may access the charting models and customize to his or her specific needs.

Furthermore, tracking module 245 can track client changes and additions to medical chart building. Although, these changes and additions will not be added to the universal charting data elements 230 when made by the client, they are tracked by the tracking module 245 and can be used for model chart building and AI learning from changes and additions during medical chart building.

Communication module 240 is in two-way communication with multiple clients 210-225 providing the medical charting build tool 205, universal charting data elements 230 and documentation rules engine 235. Typical communication module leverages a web-based application for providing data to multiple clients. Communication module 240 provides user interfaces for clients to build model charting. From these user interfaces, client chart builders can choose hard-coded charting data elements to be shown hidden to the provider based on specific patient documentation, venue and clinician specifics. Thus, as described in FIGS. 3-5, the charting interface provided to clinicians when treating patients may display certain data elements for charting while others not needed are hidden from clinician view when charting.

FIG. 3 is an illustrative screen display of universal charting data elements for vital signs that are hard-coded to the cloud-based integrated charting system. The data elements are maintained in a hierarchical structure. All hard-coded medical charting data elements for vital signs are in an event set hierarchy that creates relationships between hard-coded data elements. Primary vital signs charting data elements include secondary elements (A-D) of body temperature, pulse rate, respiration rate and blood pressure. Each of the secondary elements contains tertiary elements. For example, secondary charting data element body temperature (A) includes tertiary elements (1-5) of oral, rectal axillary, ear and skin. It is pristine content in that universal charting data elements of system can be utilized to by multiple clients to build medical charts.

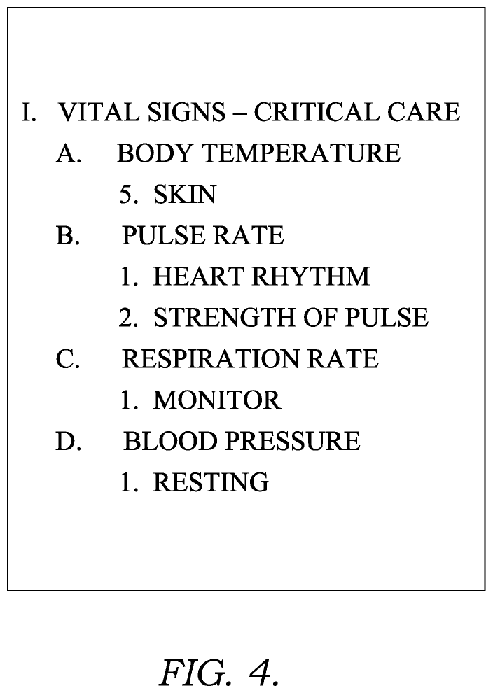

FIG. 4 is an illustrative screen display of a medical chart built by a client in a critical care department for vital signs. As illustrated in FIG. 4, the client has chosen to only display certain vital sign requests in the critical care vital sign chart form. For example, the built chart will request that a critical care provider document a skin body temperature (I.A.5.), heart rhythm and strength of pulse for pulse rate (I.B.1.2), monitor respiration (I.C.1) and resting blood pressure (I.D.1). The client builder has chosen to hide vital signs of oral, rectal, axillary and ear body temperature (I.A.1-4) as the critical care department of the client only ever takes a skin body temperature in critical care. This makes the charting display presented to the clinician more manageable, more efficient and takes up less real estate room as only one of the body temperature (e.g., skin) is selectable for charting instead of having to find the proper body temperature to chart from a list of five types.

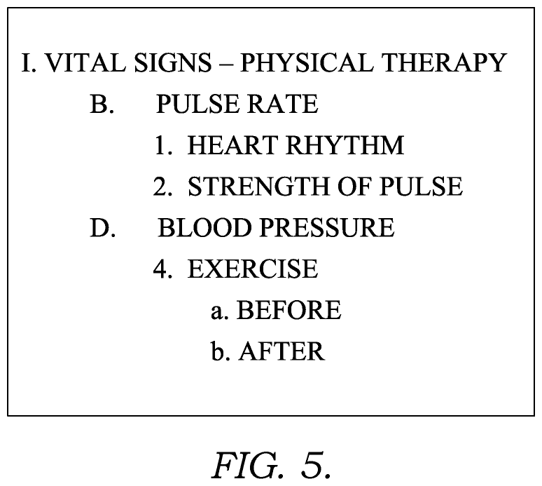

FIG. 5 is an illustrative screen display of a medical chart built by a client in a physical therapy department for vital signs. As illustrated in FIG. 5, the client has chosen to only display certain vital sign requests in the physical therapy vital sign chart. Unlike critical care environment, the physical therapy department is only going to request chart documentation of pulse rate (I.B.1-2) and blood pressure (I.D.4.a-b) when seeing a patient. The physical therapy does not typically take body temperature and respiration rates for patients. Again, this makes the charting display presented to the physical therapy clinician more manageable, more efficient and takes up less real estate room as only pulse rate and blood pressure are displayed for the clinician to chart and document.

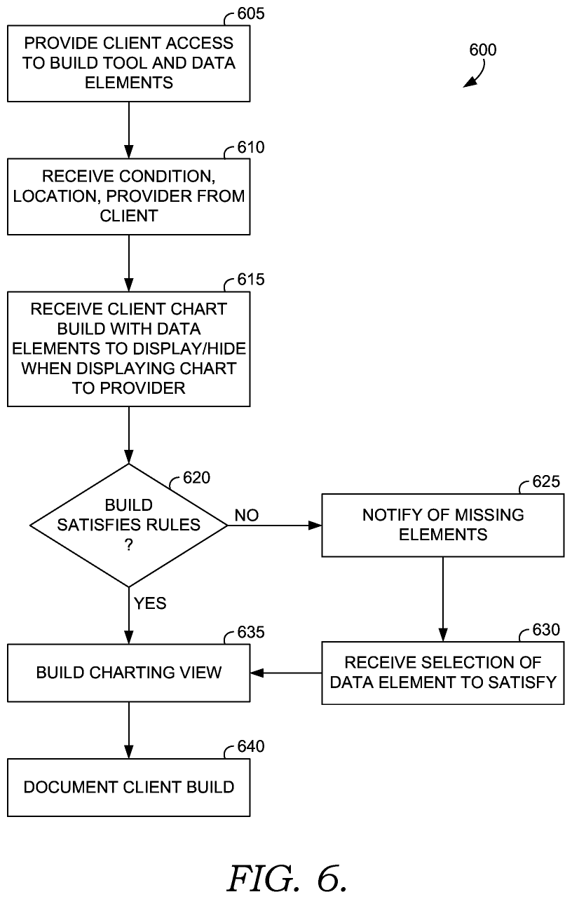

Turning now to FIG. 6, a flow diagram is provided illustrating a method 600 for determining whether a charting form built by a client satisfies conditional rules, in accordance with embodiments of the present invention. Method 600 may be performed by any computing device (such as computing device described with respect to FIG. 1) with access to a cloud-based integrated charting system (such as the one described with respect to FIGS. 2-4) or by one or more components of the cloud-based integrated charting system.

Initially, at step 605, clients are provided access to the medical charting build tool and universal charting data elements. At step 610, the client chart builder inputs a condition, location and/or provider for building a chart. For example, the chart may be for a department such as physical therapy or critical care. The chart may be for patients with specific conditions such as diabetes or heart failure. In another instance, the chart may be for preferences for a specific provider, such as a cardiologist.

At step 615, the system receives the client chart build that includes which universal data elements to display and hide when rendering the chart form to a clinician. For example, the chart builder can choose which elements to display to a user to document and which elements to hide. For example, a client builder may select addition (to include) or subtraction buttons (to hide) data elements when the chart form is displayed to a clinician. When the chart is displayed to a clinician, the clinician documents those displayed elements for the patient. Should the clinician wish to document elements not shown, the user can access those by expanding or searching for hidden data elements.

At step 620, the rules engine is applied to determine if the chart form built satisfies rules. For example, at 620 it is determined whether the build satisfies Medicaid, Medicare, insurance or meaningful use requirements for the particular condition, location or provider. The rules engine is accessed by system 200 and applies the conditionality rules. If the rules engine determines that there are data elements that will need to be documented for a patient when charting that are not included in the client built chart form, a notification is sent to the client builder at step 625.

The client builder then selects the data element to be added in order to satisfy the conditionality rule at step 630 and it is added to the chart build form. When the chart build satisfies the conditionality rules, the chart is built at step 635. When the clinician is charting for a patient based on the specified condition, location or provider, the client chart build is displayed and the user enters the appropriate values for the charting data elements. At step 640, the system 200 receives, store the client chart build form and associated conditions, location and provider for use with other clients and machine learning.

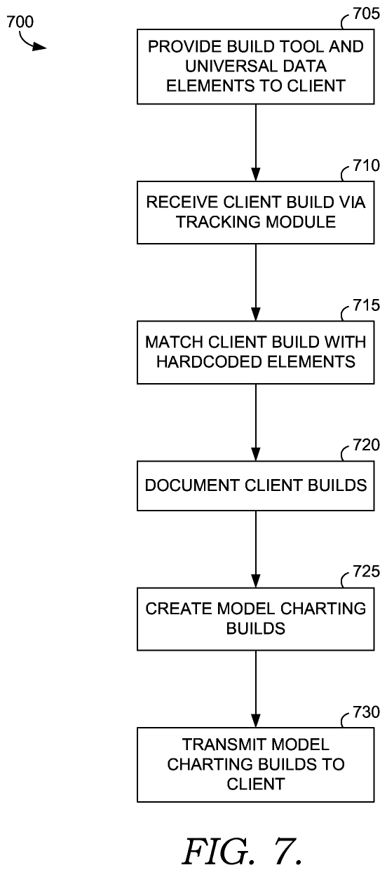

Turning now to FIG. 7, a flow diagram is provided illustrating a method 700 for building and tracking customized medical charts utilizing universal data elements common to multiple clients, in accordance with embodiments of the present invention. Method 700 may be performed by any computing device (such as computing device described with respect to FIG. 1) with access to a cloud-based integrated charting system (such as the one described with respect to FIG. 2) or by one or more components of the cloud-based integrated charting system.

Initially, at step 710, a client medical chart build built by client from the medical charting build tool and universal charting data elements at step 705 is received by system 200. The tracking module of system 200 receives the client chart build and matches the client build elements with the universal charting data elements at step 715. The system 200 stores the client chart build at step 720 and at step 725 creates model chart builds. The model chart builds can be utilized by other clients and/or departments that treat the same condition, location and/or use the same providers. The model chart builds are entered into the medical charting build tool whereby they can be transmitted to clients at step 730 to assist them with best practices for building charts by providing charting models.

Many different arrangements of the various components depicted, as well as components not shown, are possible without departing from the spirit and scope of the present invention. Embodiments of the present invention have been described with the intent to be illustrative rather than restrictive. Alternative embodiments will become apparent to those skilled in the art that do not depart from its scope. A skilled artisan may develop alternative means of implementing the aforementioned improvements without departing from the scope of the present invention.

It will be understood that certain features and subcombinations are of utility and may be employed without reference to other features and subcombinations and are contemplated within the scope of the claims. Not all steps listed in the various figures need be carried out in the specific order described. Accordingly, the scope of the invention is intended to be limited only by the following claims.

What is claimed is:

1. A computer-implemented method comprising:
receiving, by a cloud-based integrated charting system, a request from a first client device of a user to build a model chart for a medical chart associated with a patient;
accessing, by the cloud-based integrated charting system, a database cluster based at least in part on the request, wherein the database cluster comprises universal charting data elements of the model chart;
generating, by the cloud-based integrated charting system, the model chart based at least in part on the universal charting data elements and the request, wherein generating the model chart comprises:
receiving, by the cloud-based integrated charting system, configuration input from the first client device;
generating, by the cloud-based integrated charting system, first modified universal charting data elements that are intended to be displayed on the first client device by modifying a configuration of the universal charting data elements based at least in part on the configuration input; and
generating, by the cloud-based integrated charting system, second modified universal charting data elements that are intended to be displayed on the first client device by executing one or more rules on the first modified universal charting data elements, wherein the one or more rules are identified based on a regional location of the first client device;
causing to display, by the cloud-based integrated charting system, on the first client device the medical chart in accordance with the second modified charting data elements of the model chart;
storing, by the cloud-based integrated charting system, the model chart in the database cluster based at least in part on the first regional location of the first client device;
training a machine learning model of the cloud-based integrated charting system based on the model chart to obtain a refined cloud-based integrated charting system; and
causing to display, by the refined cloud-based integrated charting system, on a second client device another medical chart based at least in part on another request from the second client device at a second regional location that matches the first regional location and the model chart.

2. The computer-implemented method of claim 1, wherein the configuration input includes instructions to selectively control addition or subtraction of a subset of universal charting data elements of the universal charting data elements.

3. The computer-implemented method of claim 1, wherein at least one of the medical chart or the another medical chart are further based at least in part on a condition, a provider, or a client domain preference build.

4. The computer-implemented method of claim 1, wherein the universal medical charting elements include one or more of: vital signs, diagnoses, conditions, demographic information medications, treatment plans, progress notes, immunization dates, allergies, radiology images, and laboratory results.

5. The computer-implemented method of claim 1, wherein the universal data elements are structured in a hierarchical structure.

6. The computer-implemented method of claim 1, wherein generating the model chart further comprises:
determining, by the cloud-based integrated charting system, that the first modified universal charting data elements includes a first universal charting data element that is not associated with a department associated with the user, wherein generating the second modified universal charting data elements is further based at least in part on removing the first universal charting data element from the first universal charting data element.

7. The computer-implemented method of claim 1, further comprising:
providing, by the cloud-based integrated charting system, an alert to the first client device based at least in part on the medical chart being non-compliant with a region regulation.

8. A cloud-based system comprising:
a processor;
a database cluster that is accessible by a plurality of client devices, the database cluster including universal medical charting data elements; and
a computer storage medium storing computer-usable instructions that, when used by the processor, cause the processor to:
receive a request from a first client device of a user to build a model chart for a medical chart associated with a patient, wherein the plurality of client devices includes the first client device;
access the database cluster based at least in part on the request;
generate the model chart based at least in part on the universal charting data elements and the request, wherein generating the model chart comprises:
receive configuration input from the first client device;
generate first modified universal charting data elements that are intended to be displayed on the first client device by modifying a configuration of the universal charting data elements based at least in part on the configuration input; and
generate second modified universal charting data elements that are intended to be displayed on the first client device by executing one or more rules on the first modified universal charting data elements, wherein the one or more rules are identified based on a regional location of the first client device;
cause to display on the first client device the medical chart in accordance with the second modified charting data elements of the model chart;
store the model chart in the database cluster based at least in part on the first regional location of the first client device;
train a machine learning model of a cloud-based integrated charting system based on the model chart to obtain a refined cloud-based integrated charting system; and
cause to display, based on an output from the refined cloud-based integrating charting system, on a second client device another medical chart based at least in part on another request from the second client device at a second regional location that matches the first regional location and the model chart, wherein the plurality of client devices includes the second client device.

9. The cloud-based system of claim 8, wherein the configuration input includes instructions to selectively control addition or subtraction of a subset of universal charting data elements of the universal charting data elements.

10. The cloud-based system of claim 8, wherein at least one of the medical chart or the another medical chart are further based at least in part on a condition, a provider, or a client domain preference build.

11. The cloud-based system of claim 8, wherein the universal medical charting elements include one or more of: vital signs, diagnoses, conditions, demographic information medications, treatment plans, progress notes, immunization dates, allergies, radiology images, and laboratory results.

12. The cloud-based system of claim 8, wherein the universal data elements are structured in a hierarchical structure.

13. The cloud-based system of claim 8, wherein generating the model chart further comprises:
determining, by the cloud-based integrated charting system, that the first modified universal charting data elements includes a first universal charting data element that is not associated with a department associated with the user, wherein generating the second modified universal charting data elements is further based at least in part on removing the first universal charting data element from the first universal charting data element.

14. The cloud-based system of claim 8, further comprising:
providing, by the cloud-based integrated charting system, an alert to the first client device based at least in part on the medical chart being non-compliant with a region regulation.

15. A non-transitory computer readable medium having instructions that, when executed by a processor, cause the processor to:
receive a request from a first client device of a user to build a model chart for a medical chart associated with a patient;
access a database cluster based at least in part on the request, wherein the database cluster comprises universal charting data elements of the model chart;
generate the model chart based at least in part on the universal charting data elements and the request, wherein generating the model chart comprises:
receive configuration input from the first client device;
generate first modified universal charting data elements that are intended to be displayed on the first client device by modifying a configuration of the universal charting data elements based at least in part on the configuration input; and
generate second modified universal charting data elements that are intended to be displayed on the first client device by executing one or more rules on the first modified universal charting data elements, wherein the one or more rules are identified based on a regional location of the first client device;
cause to display on the first client device the medical chart in accordance with the second modified charting data elements of the model chart;
store the model chart in the database cluster based at least in part on the first regional location of the first client device;
train a machine learning model of a cloud-based integrated charting system based on the model chart to obtain a refined cloud-based integrated charting system; and
cause to display, based on an output from the refined cloud-based integrating charting system, on a second client device another medical chart based at least in part on another request from the second client device at a second regional location that matches the first regional location and the model chart.

16. The non-transitory computer readable medium of claim 15, wherein the configuration input includes instructions to selectively control addition or subtraction of a subset of universal charting data elements of the universal charting data elements.

17. The non-transitory computer readable medium of claim 15, wherein at least one of the medical chart or the another medical chart are further based at least in part on a condition, a provider, or a client domain preference build.

18. The non-transitory computer readable medium of claim 15, wherein the universal medical charting elements include one or more of: vital signs, diagnoses, conditions, demographic information medications, treatment plans, progress notes, immunization dates, allergies, radiology images, and laboratory results.

19. The non-transitory computer readable medium of claim 15, wherein the universal data elements are structured in a hierarchical structure.

20. The non-transitory computer readable medium of claim 15, wherein generating the model chart further comprises:
determining that the first modified universal charting data elements includes a first universal charting data element that is not associated with a department associated with the user, wherein generating the second modified universal charting data elements is further based at least in part on removing the first universal charting data element from the first universal charting data element.
